(19) United States
(12) Patent Application Publication
(10) Pub. No.: US 2023/0107635 A1
(43) Pub. Date: Apr. 6, 2023

(54) CRISPR ASSAY FOR RAPID, ENHANCED SCREENING OF HPV-RELATED DISEASE

(71) Applicant: OncoGenesis, Inc., Salt Lake City, UT (US)

(72) Inventors: Paul VICHI, Lincoln, VT (US); Peter P. GOMBRICH, Salinas, CA (US)

(21) Appl. No.: 17/956,078

(22) Filed: Sep. 29, 2022

Related U.S. Application Data
(60) Provisional application No. 63/251,221, filed on Oct. 1, 2021.

(51) Int. Cl. G01N 33/574; G01N 33/569; C12Q 1/6844

(52) U.S. Cl. CPC G01N 33/57411; G01N 33/56983; C12Q 1/6844; G01N 2333/025; G01N 2333/4742

(57) ABSTRACT

The present disclosure provided methods, devices, and systems for CRISPR-based screening and detection of HPV-related diseases. In particular, the present disclosure provides a CRISPR-Cas assay for rapid, enhanced screening of cervical intraepithelial neoplasia (CIN) and cancer, which can also be applied to screening for other HPV-related anogenital or head and neck cancers, whose origin is based on infections with high-risk strains of human papillomavirus (hr-HPV).

### CROSS-REFERENCE TO RELATED APPLICATIONS

This application claims benefit of U.S. provisional patent application Ser. No. 63/251,221, filed Oct. 1, 2021, which is herein incorporated by reference in its entirety.

### BACKGROUND

### Field

Embodiments of the present disclosure generally relate to methods, devices, and systems for detection of cervical disease, and more particularly, point-of-care (POC) CRISPR-based screening of human papillomavirus (HPV) and HPV-associated cervical disease.

### Description of the Related Art

Cervical cancer is one of the most common and preventable cancers affecting women worldwide, accounting for roughly 300,000 deaths each year, primarily in developing regions. Cervical disease is the result of persistent infection with high-risks strains of the human papilloma virus (HPV), a common sexually transmitted disease. While most women are able to clear the virus through normal immune function, a subset of women will exhibit persistent infection leading to transformation of precancerous lesions, which may develop into cervical cancer.

Current methods to screen for cervical cancer rely on subjective cytology to microscopically identify abnormal cells in patient cervical specimens, and more recently, molecular tests to identify the presence of HPV, the etiological agent for cervical, as well other anogenital and head and neck cancers. Cytology requires highly trained personnel and significant infrastructure, and often takes 1 to 2 weeks for completed results. While HPV screening is useful to identify patients that present with an infection and can run on higher throughput clinical laboratory devices, it requires expensive instrumentation and more importantly, does not provide information on disease status, which necessitates co-testing with cytology or biopsy for pathology. Moreover, the high prevalence of HPV infections in young women and the ability for many patients to successfully combat the infection reduces the power of HPV testing as a stand-alone screen to identify women at risk for cervical neoplasia or cancer, resulting in added tests, costs, time and burden.

Accordingly, what is needed in the art are improved methods and devices for POC screening of HPV-associated cervical disease.

### SUMMARY

The present disclosure generally relates to methods, apparatus, and systems for detection of cervical disease. More particularly, the present disclosure provides point-of-care CRISPR-based screening of human papillomavirus (HPV) and HPV-associated cervical disease.

Aspects of the present disclosure provide a rapid means for detection of high-risk HPV (hr-HPV) infections and evidence of HPV-related disease in patient samples that can be completed at the point-of-care (POC), thereby improving convenience, cost, and time-to-results. For example, embodiments described herein include: a cervical specimen collector for self-collection; a sample vial configured to accept a cervical specimen and containing a solution to prepare the specimen; a test vial or cartridge configured to further process the specimen and provide means for results detection, wherein the test vial or cartridge comprises a matrix, membrane, and/or microfluidic channel(s) within a housing or cartridge that contains specific chambers and reagents facilitating sample processing and results detection; a portable device compatible with the cartridge or membrane that is capable of supporting sample processing, signal detection, results reporting, sample tracking, and electronic recording of data; and a software application for use with portable devices such as smart phones, tablets, other mobile devices, laboratory instruments, and computers, etc., that provides a user interface and supports test calibration, processing, result interpretation, and reporting, logistics, etc.

Certain aspects leverage: the use of surface-modified polymeric and/or magnetic beads for capture/enrichment of target sequences; robust, low-cost, isothermal methods for target amplification such as LAMP/RT-LAMP, recombinase polymerase amplification (RPA), nucleic acid sequence-based amplification (NABA), helicase-dependent amplification (HDA), strand displacement amplification (SDA), exponential amplification (EXPAR), rolling circle amplification (RCA), and nicking extension amplification reaction (NEAR); and clustered regularly interspaced short palindromic repeats (CRISPR) and CRISPR-associated (Cas) technology for detection of target species to formulate a CRISPR-Cas assay for rapid, enhanced screening of cervical intraepithelial neoplasia (CIN) and cancer. The assay may be utilized to detect multiplexed nucleic acid biomarkers indicative of infection with hr-HPV and transformation of host cells resulting in precancerous cervical intraepithelial neoplasia and/or cervical cancer.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates a schematic top view of an example dipstick and housing for initial binding of nucleic acids from a specimen vial containing lysed cervical cells in chaotropic solution, and for transfer of the nucleic acids to reaction chambers in a test cartridge for amplification and detection.

FIG. 1B illustrates a schematic side view of an example dipstick and housing for transfer of nucleic acids to a test cartridge, showing electrode connections for reaction chamber heating, as well as the posterior guide structure and rupture tip of the housing that ensures proper orientation and rupture of internal buffer storage compartment upon insertion.

FIGS. 2A-2C illustrate schematic top and side views of an example vial and test cartridge showing entry/guide slots connecting inserted dipsticks to chambers for rinsing or amplifying and detecting target molecules. FIG. 2A illustrates the top view showing dual entry ports; FIGS. 2B and 2C illustrate opposing side views showing an example single rinse chamber and example dual reaction chambers for amplifying DNA or RNA targets.

FIG. 3 illustrates a top view of an example lateral flow reporter dipstick for insertion and result detection following amplification and CRISPR-Cas detection operations. The dipstick contains specific zones for results assessment, which can be further interpreted/transferred using image capture with an accompanying product software application for mobile, smart, or computer devices.

FIG. 4 illustrates an exemplary workflow for CRISPR/Cas detection of cervical cancer biomarkers utilizing a cartridge as described elsewhere herein. The operations of the exemplary workflow commence with sample transfer to the cartridge and conclude with the reporting of results to a user. With the exception of sample input, all steps may be automated, as facilitated by an accompanying instrument.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure provided methods, devices, and systems for CRISPR-based screening and detection of HPV-related diseases. In particular, the present disclosure provides a CRISPR-Cas assay for rapid, enhanced screening of cervical intraepithelial neoplasia (CIN) and cancer, which can also be applied to screening for other HPV-related anogenital or head and neck cancers, whose origin is based on infections with high-risk strains of human papillomavirus (hr-HPV).

Clustered regularly interspaced short palindromic repeats (CRISPR) and CRISPR-associated (Cas) systems provide a type of adaptive immunity in prokaryotes against bacteriophage or plasm ids, and technology based on these systems has been used to develop a variety of molecular tests and procedures for detection of infections such as SARS-CoV-2, or to accomplish sequence specific genome editing, base editing, or gene regulation to inactivate or correct disease-causing genes. Here, the present disclosure provides a CRISPR assay system for the rapid screening and detection of infections involving high-risk HPV strains as well as determining evidence of disease resulting from infection with hr-HPV.

CRISPR-Cas systems are comprised of multiple components including enzymatic proteins (Cas), crRNA and transactivating RNA (trRNA) sequences (combined form referred to as single guide RNA (sgRNA) or guide RNA (gRNA)), which along with clustered nucleic acid repeats and in some cases protospacer adjacent motifs (PAMs), facilitate the sequence specific recognition, binding, and activation of Cas enzyme activity at specific nucleic acid sequences of target sequences.

Cas proteins can be divided into two primary classes, each with 3 types and a variety of subtypes/homologues, which influence riboprotein complex function, whether Cas endonuclease activity is directed toward double stranded DNA or single stranded RNA (ssRNA), whether cuts are made in a blunt or staggered fashion, and whether or not the complex requires PAM for efficient binding to target nucleic acids. For example, Cas9 originating from S. pyogenes is part of a 4-component system that recognizes DNA in the context of a PAM, introducing blunt, double-stranded DNA breaks. Types IIa and IIc Cas9s however, bind and cleave ssRNA without a PAM requirement. Similarly, Cas13 from L. shahii is an RNA guided RNA endonuclease that cleaves ssRNA. Cas12a, from F. novicida, recognizes and cuts specific DNA sequences in a staggered manner requiring only crRNA for efficient targeting as opposed to both crRNA and trRNA as required by Cas9. And unlike Cas9, but similar to Cas13, Cas12a remains bound to the DNA or RNA molecule following endonuclease function, allowing non-discriminate or collateral cleavage of other ssDNA or ssRNA targets, which among other differences in riboprotein complex function, can be leveraged for diagnostic assay development.

Embodiments of the present disclosure address concerns regarding the limited clinical information that can be obtained in a single test for HPV infection or disease, as well as costs, time-to-results, and infrastructure needed to complete patient screening using standard methodology. Utilizing a blend of rapid isothermal amplification tools with CRISPR-Cas technology and either lateral flow, fluorescent detection, or electrochemical detection, the screening assays and systems described herein can detect multiplex nucleic acid signatures (RNA and/or DNA), providing patients with rapid, POC screening that detects both evidence of hr-HPV infection as well as objective evidence of disease resulting from infection with hr-HPV in a single, convenient, and easy to use format, thereby reducing time and costs to results.

By utilizing CRISPR-Cas systems that include specific crRNA, trRNA, or sgRNA directed toward defined nucleic acid sequences present in target DNA or mRNA, various Cas enzyme homologs, isothermal amplification tools such as recombinase polymerase amplification (RPA), reverse transcriptase-RPA (RT-RPA), or loop mediated isothermal amplification (LAMP/RT-LAMP) with primers to amplify multiple target species, and molecular probes whose signal is activated upon CRISPR-Cas directed cleavage such as RNA or DNA endonuclease targeted CRISPR trans reporter technology (DETCTR/OR-DETCTR), specific high sensitivity enzymatic reporter unlocking assay (SHERLOCK/OR-SHERLOCK), or quenched fluorescent ssDNA reporter such as HEX-N12-BHQ1, the present disclosure contemplates a one-pot assay that rapidly amplifies and detects specific host and viral mRNA biomarker transcripts indicative of hr-HPV strains and viral oncogene production to provide evidence of infection as well reflecting cervical intraepithelial neoplasia or cancer.

Current cervical cancer screening techniques analyze cervical cellular specimens for both the presence of infection with hr-HPV, typically through PCR directed amplification of HPV DNA, as well as co-testing of hr-HPV positive patients with cytology to determine evidence of abnormal cells or precancerous lesions. Although HPV DNA indicates patient infection, it lacks clinical information for disease status without subsequent co-testing. The use of nucleic acid biomarkers, including viral oncogenes or host gene expression, can provide additional clinical information helpful to define the presence of disease and patient risk. However, this requires timely and expensive molecular testing within a laboratory format, limiting its implementation.

In the certain embodiments, the CRISPR-Cas-enabled assay detects both hr-HPV DNA as well as viral oncogene and/or host gene expression to identify patients with hr-HPV infections and related disease in a single, low-cost portable format. The assay may employ a plurality/variety of Cas enzymes (e.g., Cas 9, 12, 12b, 13, 13b, variants thereof, and others), sgRNAs (crRNA/trRNA) primers that display activity towards target DNA or RNA molecules, and isothermal amplification methods such as RPA, RT-LAMP, NASBA, etc. Upon successful amplification, multiplex detection of amplicons is accomplished through use of Cas enzymes and crRNA or sgRNA to ensure specific target recognition/binding and activation of specific and/or non-specific Cas endonuclease function against accompanying colorimetric or fluorescent labeled reporter probes, whose signal can be visualized on lateral flow strips, portable benchtop instruments, hand-held readers (e.g., smartphones, tablets, computers, etc.), and other devices for rapid determination. Accordingly, the systems described herein are portable, low cost, field- or clinical-deployable, and may provide results in about one hour or less.

Nucleic acid targets for hr-HPV detection include viral DNA sequences present in L1 and/or L2 genes, which code for the major and minor viral capsid proteins and can be used to identify infection resulting from up to 15 hr-HPV strains, including HPV 16, HPV 18, HPV 31, HPV 33, HPV 35, HPV 39, HPV 45, HPV 51, HPV 52, HPV 53, HPV 56, HPV 58, HPV 59, HPV 66, and HPV 68 in patient specimens. Nucleic acid targets for indication of HPV-induced disease include viral mRNA transcripts for specific HPV oncogenes such as E6 and or E7, which may be elevated following persistent infection and viral integration into the host cell genome, as well as several patient cell mRNA transcripts, the level of which may be directly or indirectly related to the presence of up-regulated viral oncogenes and are associated with transformation in the context of persistent hr-HPV. These include, but are not limited to, transcripts for p16, ERK-1, hTert, LR-67, MMP-2, Nf-Kβ, nm23-H1, PCNA, survivin, Topo-2α, VEGF-C, cytokeratin 17 (K17), etc. Therefore, whereas determination of hr-HPV infection relies on identifying viral DNA sequences, disease status and/or prognostic risk in HPV-positive patients relies on detecting specific mRNA transcripts, typically necessitating multiple assays to achieve. Embodiments described herein offer the option of rapid detection for oncogenic expression in cervical specimens, including the potential detection of both DNA and RNA markers for cervical disease within a single test. Furthermore, the detection of multiple biomarkers in the same assay provides greater clinical information assisting in the determination of risk, progression, and subsequent treatment options not obtainable with current procedures.

In certain embodiments, the system provides for ease of use in terms of sample preparation, such as but not limited to: the lysis of cellular specimens and the capture/isolation of nucleic acid targets on filters, or surface-modified polymeric or magnetic beads; isothermal amplification of target RNA and/or DNA; as well as detection schemes involving Cas RNA-guided sequence recognition (with or without PAM requirements), DNA and/or RNA cleavage activity, and multiplex qualitative or semi-quantitative signal detection for target biomarkers and system control(s).

Lysis of cervical cells or other cellular based specimens can be achieved through a variety of methods, provided the methods preserve the quality/quantity of nucleic acids for subsequent activities. Cellular lysis methods may include, but are not limited to, physical disruption such as mechanical shearing and applied or reduced pressure; thermally induced changes such as sample heating, freezing, and cavitation; chemical means such detergents, and chaotropic, aqueous, hypotonic, or phenolic agents; enzymatic methods; or a combination thereof, e.g., a hypotonic solution and mechanical shearing. In certain embodiments, the lysis is achieved in a neutral to weakly alkaline chaotropic solution that supports the stability of both DNA and RNA, such as a guanidine hydrochloride or guanidinium isothiocyanate—Tris-based reagent containing Triton and/or Tween detergents, with or without proteinase K.

In certain embodiments, nucleic acid binding, capture, and/or isolation is achieved using membranes, dipsticks, polymeric or magnetic beads etc., designed to bind or trap nucleic acids on a portion of the dipstick as a function of pore size, charge, or material or complementary molecular nature. Several materials are known to efficiently bind nucleic acids, including cellulose, nitrocellulose, silicas, paramagnetic silicas, diatoms, polyethersulfone (PES), nylon, etc. Material surfaces may be selected or further modified for hydrophilic or hydrophobicity properties, and/or to enhance nucleic acid capture via electrostatic or ionic binding, or via complementarity, such as poly dT to enrich capture of polyadenylated mRNA.

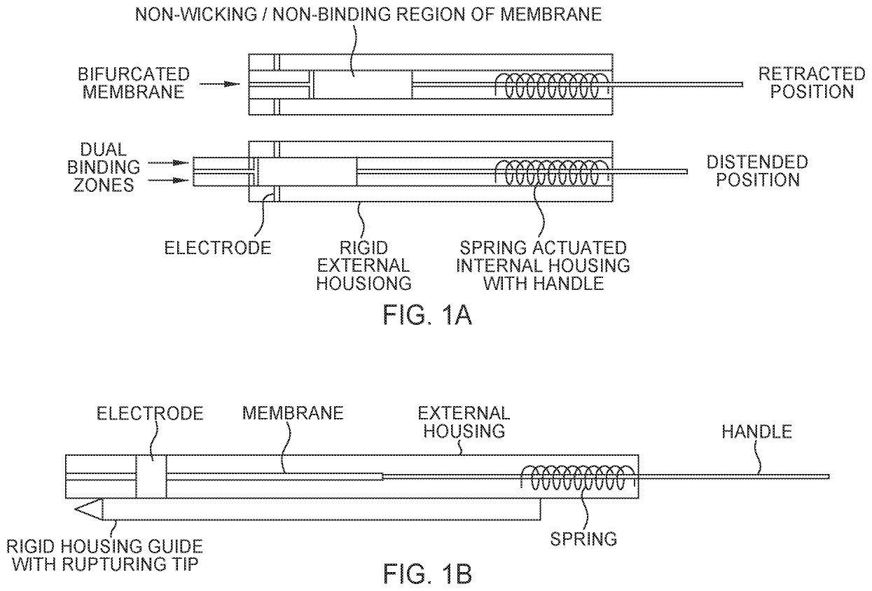

In certain embodiments, a dipstick is bifurcated at the capture zone and is designed/modified such that one side optimally binds/captures DNA and the other side enriches mRNA. FIG. 1A illustrates a schematic top view of an example bifurcated dipstick and its corresponding housing for initial binding of nucleic acids from a specimen vial containing lysed cervical cells in chaotropic solution, and for transfer of the nucleic acids to reaction chambers in a test cartridge for amplification and detection. The dipstick is shown in both the retracted and distended positions, which are made possible through activation of a spring-loaded handle on the distal end of the unit relative to the collection/binding zone of the membrane. In certain embodiments, the dipstick is further modified to ensure capture of target material to a defined section, the remainder housed within support structure and modified to prevent movement or binding of material beyond the active zone. To limit the transfer of non-target material that may reduce the efficiency of target species amplification or detection, the dipstick containing bound nucleic acids is washed briefly in a sterile aqueous solution containing Tris, +/−Tween and MgCl prior to downstream use. In other embodiments, the capture of mRNA is facilitated by magnetic poly-dT beads, the movement of which can be controlled through instrument/cartridge driven processes.

In certain embodiments, the dipstick is configured to fit within a thin, rigid housing such that the full area for binding is exposed while the upper region is held within the housing. The housing is further configured to fit within a test vial or cartridge that contains multiple reaction chambers for rinsing, target amplification, and CRISPR-Cas mediated detection, such that when fully inserted, the edges of the housing contact a stop in the vial/cartridge wall, thus preventing further movement. In certain embodiments, the stop in the vial or cartridge is located such that the exposed tip of the dipstick is fully inserted in the rinse or reaction solutions present in the chamber(s). The filter housing and vial or cartridge opening may be arranged such that the vial or cartridge can only accept the filter housing in one direction, thereby maintaining positional/molecular differences obtained from a bifurcated filter tip in subsequent reactions. In certain embodiments, the sample is manually transferred and subsequently contained/processed within a reaction chamber housed within a multi-layered microfluidic cartridge. The microfluidic cartridge may be pre-deposited with specific reagents in lyophilized or liquid form, as necessary to support assay steps, such as lysis, capture, amplification, detection, etc.

In certain embodiments, such as the example of FIG. 1A, the distal end of the dipstick housing, relative to the binding region, is constructed with a spring handle design such that by depressing the top or proximal end of the dipstick housing, the distal end of the filter end can be further distended into the rinse or reaction tubes ensuring the filter tip makes contact with the bottom of the chamber, resulting in deformation of the tip and improving rinsing or elution efficiency. Dipsticks with bound nucleic acids can be rinsed to remove contaminants by sliding the dipstick housing into the vial/cartridge opening until the housing reaches the internal stop and then depressing the spring handle several times to fully rinse the filter tip in the supplied buffer. In other cartridge-based embodiments, the target species for amplification are captured on surface-modified magnetic beads present within a lysis chamber to which the specimen is introduced. Manipulation of the beads may be accomplished via portable instrumentation that accepts the cartridge.

The housing containing the rinsed filter tip may then be transferred to a second opening in a vial or cartridge containing a lyophilized reaction pellet with all necessary oligonucleotide primers, reagents, etc., for multiplex target amplification. Amplification of target sequences for detection can be completed using a variety of methods such as PCR, LAMP/RT-LAMP, or RPA. For purposes of this disclosure, low-cost, robust, rapid processes with minimal required manipulation are preferred. Furthermore, processes that can be completed at lower isothermal temperatures and directed at both RNA and DNA can be used to avoid demands associated with thermocycling PCR-based amplifications.

To facilitate amplification reactions or other reactions that require the introduction of buffer to solubilize lyophilized material, the edges of the dipstick housing may be designed as a rigid tip suitable for rupture of sealing membranes, such that when fully inserted into a test vial or cartridge, the tip ruptures a thin sealing membrane initiating buffer flow into the reaction chamber. An exemplary dipstick having a rigid housing with a rigid tip for rupturing is depicted in FIG. 1B. In addition to facilitating rupturing of an internal buffer storage compartment of a cartridge or test vial upon insertion of the dipstick, the rigid housing may also serve as a guide to ensure proper orientation of the dipstick during insertion. As further shown, the dipstick may comprise an electrode for coupling with a reaction chamber heating source to heat a captured sample. For example, in a cartridge-based embodiment, portable instrumentation may provide the required energy/heat for amplification, as well as introduction and removal of buffers and other fluid reagents. Reaction chamber(s) containing required reagents in lyophilized form that can be resuspended/mixed upon internal or external fluid sample introduction may be controlled via the instrument.

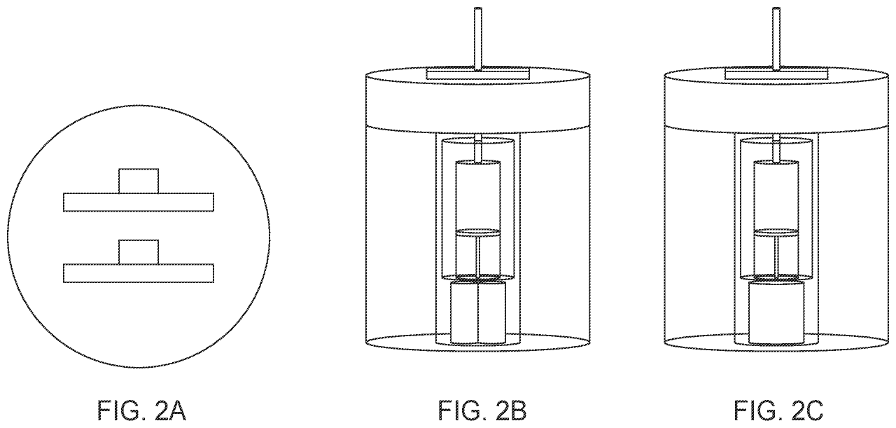

FIGS. 2A-2C illustrate schematic top and side views of an example vial with a test cartridge therein showing entry/guide slots of the vial connecting inserted membranes or dipsticks to chambers for rinsing or amplifying and detecting target molecules. FIG. 2A illustrates a top view of a vial showing openings for a membrane or dipstick and housing unit with a primary opening and guide slot ensuring insertion of a membrane/dipstick in a single, correct orientation. FIGS. 2B and 2C illustrate opposing side views of an exemplary vial having a housing unit with dual reaction chambers for amplification and detection of DNA and RNA markers, and an exemplary vial having a housing unit with single rinse chamber, respectively. The caps of these vials may comprise one or more openings which are connected to reaction chambers positioned below the caps. For rinsing, a vial may comprise one larger chamber, as shown in FIG. 2C; for amplification and detection, a two vial may comprise two chambers that preferentially amplify and detect DNA versus RNA species, as shown in FIG. 2B. A button cell battery located in the base of a vial may provide power to the reaction chambers for heating.

Certain embodiments of the present disclosure utilize low-temperature, isothermal means to amplify targets, as provided by RPA/RT-RPA or LAMP/RT-LAMP, which can be performed at 37° C.-42° C. The use of a bifurcated membrane for nucleic acid binding enriched for DNA or RNA molecules is further exploited in the amplification step by ensuring that DNA or RNA molecules are deposited into specific reaction chambers in the vial or cartridge that are optimized for target DNA or RNA amplification and that further maintain positional differences for amplification and subsequent detection via lateral flow or direct spectrophotometric examination. In a microfluidic cartridge-based embodiment, the binding and introduction of nucleic acids to a cartridge chamber for amplification may be facilitated by nucleic acid capture on surface-modified magnetic beads, which may be manually transferred to the cartridge reaction chamber using a fixed-volume pipette.

Sterile buffer can be introduced to the reaction chambers containing lyophilized material upon insertion of the dipstick, which ruptures a sealant membrane, initiating flow into the reaction chamber. Nucleic acids are then eluted from the dipstick through repetitively depressing the spring handle of the dipstick while it is disposed in the reaction chamber, which distends the tip sufficiently to contact the solution and the bottom of the chamber maximizing elution. Whereas the resting position of the membrane tip is fully within the available solution when the housing is inserted into the vial/cartridge for binding or rinsing steps, in certain embodiments, for the deposition step into the amplification reaction, the resting position of the membrane tip remains just above the surface of the reaction solution when the housing is fully inserted in the vial/cartridge and the spring handle is not depressed. Only when fully depressing the spring-loaded section of the housing will the membrane contact the solution. The described configuration ensures time for solution heating before elution and removes the membrane once the elution process has been completed.

In certain embodiments, heating of the reaction fluid to the desired temperature is facilitated by a disposable battery-powered heating element and thermistor connected to the amplification reaction chambers. Heating of the reaction chambers for amplification may be activated following insertion of the dipstick housing into the amplification opening of the vial/cartridge, which upon contact with the stop position completes the necessary circuit to initiate rapid heating of the solution(s) to the desired temperature. Removal of the housing breaks the circuit and discontinues any further heating.

In certain embodiments, detection of amplified targets is performed in the same reaction chamber used for target amplification via inclusion therein of all necessary CRISPR-Cas enzymes, cr- and/or trRNAs and reporters, designed to recognize target biomarkers and produce detectable signal. A plurality of Cas enzymes provides options for detection of DNA or RNA molecules using specific Cas enzymes that recognize nucleic acid sequences in DNA or RNA and can cleave collateral or non-specific DNA or RNA probes. For example, target RNA species may be detected via inclusion of Cas13, which is an RNA-guided RNA endonuclease that remains bound to target and cleaves collateral ssRNA probes. Inclusion of Cas12a in the reaction can facilitate detection of target DNA biomarkers and cleavage of ssDNA probes. Furthermore, the use of multiple chambers and reporters within the vial that accepts the bifurcated filter tip ensures that specific chambers can be used to detect distinct biomarkers with DNA or RNA origin, and the result of each reaction can be further delineated on lateral flow or spectrophotometric detection steps. For detection using lateral flow, the membrane housing containing the filter used to introduce nucleic acids to the reaction may be removed and a new housing containing a lateral flow detection strip can be inserted into the same opening. The lateral flow strip and housing facilitate solution uptake through the exposed end of the strip into the body of the strip and the housing contains openings within the length of the body at which signals may be interpreted or visualized.

Reporter probes may be designed with both quencher and fluorophores or means for colorimetric reporting, and secondary structures such as hairpins, etc., ensuring no signal is emitted until Cas directed cleavage occurs. Upon cleavage, the fluorophore or colorimetric signal agent is emitted or freed from bound constraint and can be detected by a variety of means, such as spectrophotometric analysis of the reaction solution or via lateral flow and visualization. In lateral flow, signals are directly visualized or directly interpreted off of the membrane strip. In a cartridge/instrument-based approach relying on fluorescence or electrochemical signals, detection of target species is accomplished through instrument sensing of the reporter molecules resulting from Cas enzyme cleavage within the reaction chamber as targets are amplified, thereby providing real-time detection.

Reporters for lateral flow detection may be further configured to contain components such as biotin or digoxigenin (DIG) for binding to streptavidin, anti-DIG molecules or immunoglobulin or binding moiety to interact with signal molecules that migrate through the lateral flow strip, or preloaded molecules present at windows of the housing on lateral flow strips to provide a positionally-configured trap for site-specific concentration and visualization of reporter molecules. Different binding components can be used with different signal molecules e.g., biotin versus DIG-linked fluorophores, to distinguish signal origin for multiplex applications, and position (lane) on the lateral flow membrane can be used to distinguish control versus positive or negative presence of specific target biomarkers.

When using specific RNA-directed CRISPR Cas enzymes, controls for contaminating RNase activity may be warranted. These can be included within the detection reaction and differentiated in lateral flow through specific signal emission and positional reference on the lateral flow strip. For example, inclusion of Cas13a-resistant reporters with specific labels that remain susceptible to RNase I-, RNase T1- or RNase A-mediated cleavage can be used to differentiate signal from RNA targets cleaved by Cas13a versus contaminating RNase activity. Additional controls may also be included as separate lanes on lateral flow or within reaction chambers to provide internal sample controls to verify sample adequacy, input, or test accuracy (+/−controls). These controls may be represented by distinct DNA or mRNA, which do not vary in level as a function of HPV-related disease; and provide information on the level of control. Sample controls may include, but are not limited to, one or more targets such as B-actin, GAPDH, cytokeratin 5, cytokeratin 8, cytokeratin 18, etc.

Results detection and interpretation following lateral flow may be further enhanced/processed by image capture and analysis using an accompanying product application compatible with mobile devices or computer. In certain embodiments, image capture is accomplished through use of native camera, and analysis via an embedded algorithm within the application. The application may provide a user with a concise report regarding the results, and may even suggest a follow up screen or test if necessary. The application may also provide the user with product information, instructions for use, customer service, etc. The results are compatible with telemedicine practice, and the application can transmit test data to a patient's physician or a remote clinic for review and further consultation.

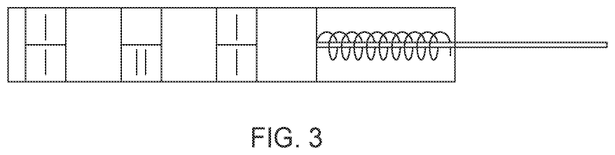

FIG. 3 illustrates a top view of an example lateral flow reporter dipstick for insertion and result detection following amplification and CRISPR-Cas detection operations. The dipstick contains specific zones for results assessment, which can be further interpreted/transferred using image capture with an accompanying product software application for mobile, smart, or computer devices. Visualization of the results following amplification and CRISPR-Cas-mediated detection in the presence of reporter molecules may be facilitated with introduction of the depicted reporter dipstick. In FIG. 3, the results are represented as lines, which are observed within designated regions of the lateral flow dipstick. The DNA results and controls may be present on one side of the membrane (top), and RNA markers and controls visualized on the opposing side (bottom). An image of the results can be captured with an accompanying software application for mobile devices and interpreted for ease of conclusions to the viewer.

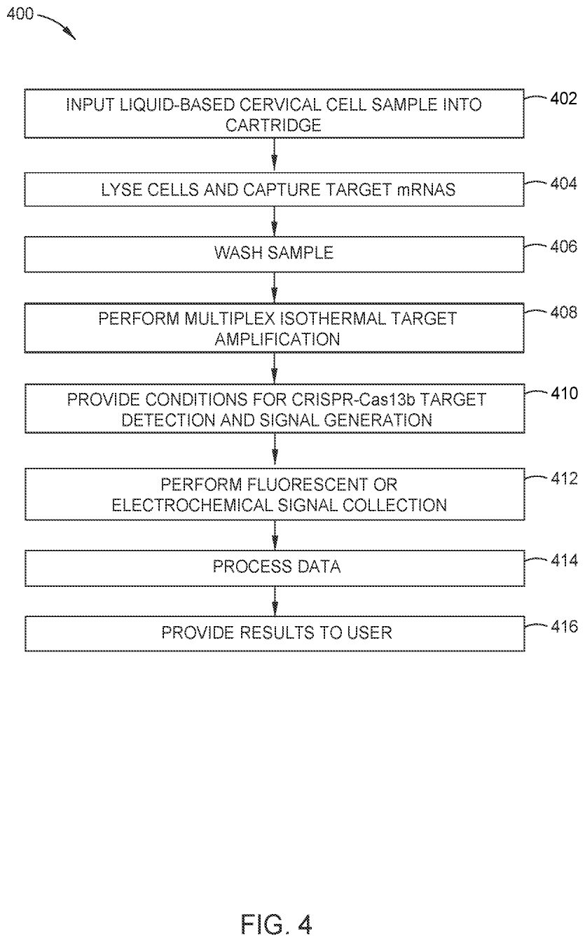

FIG. 4 illustrates an exemplary workflow 400 for CRISPR/Cas detection of cervical cancer biomarkers utilizing a cartridge-based system as described elsewhere herein. The operations of the exemplary workflow commence with sample transfer to the cartridge and conclude with the reporting of results to a user. With the exception of sample input, all steps may be automated, as facilitated by an accompanying instrument.

As shown in FIG. 4, the workflow 400 begins at operation 402, wherein a cervical cell sample, in liquid, is transferred to a test cartridge. In certain embodiments, the test cartridge is a microfluidic cartridge with defined ports, conduits, and chambers for sample input, flow, processing, and results presentation. The test cartridge may comprise all the necessary reagents/materials in lyophilized or liquid formats to facilitate sample lysis, capture/isolation of target nucleic acids, isothermal amplification, CRISPR detection, and reporting. The cartridge is supported in function by a portable instrument supplying necessary power, pressure, heating/cooling, signal collection/reporting and calibration, data management.

At 404, the cervical cell sample, in liquid, is lysed via any suitable means as described herein (e.g., chemical lysis), and target mRNAs (e.g., for specific HPV oncogenes) from the lysed sample are captured on a suitable substrate. In certain embodiments, the mRNAs are captured on modified magnetic beads, such as poly-dT-magnetic beads.

At 406, the test cartridge is introduced into an accompanying instrument, e.g., a portable benchtop instrument that provides power, processing support, and a user interface, and the substrate comprising the anchored target nucleic acids is washed. The instrument may include any suitable system for providing control of sample/stage residence time, temperature, buffer inputs, sample removal, reagent flow, and the like, within the test cartridge.

At 408, the target nucleic acids are amplified using any suitable multiplex methods, such as PCR, LAMP/RT-LAMP, or RPA. Accordingly, the test cartridge may comprise, or the instrument may provide to the test cartridge, all necessary oligonucleotide primers, reagents, etc., for multiplex target amplification.

At 410, conditions for multiplex detection and signal generation of target amplicons are provided. Target detection and signal generation is facilitated through use of Cas enzymes, e.g., Cas13b, and crRNAs or sgRNAs to ensure specific target recognition/binding and activation of specific and/or non-specific Cas endonuclease function against accompanying reporter probes. In certain embodiments, detection of amplified targets is performed in the same reaction chamber of the test cartridge used for target amplification via inclusion therein of all necessary CRISPR-Cas enzymes, cr- and/or sgRNAs and reporters, designed to recognize target biomarkers and produce detectable signal.

At 412, signal collection is performed. In a cartridge/instrument-based approach relying on fluorescence or electrochemical signals, such as the example described herein, detection of target species is accomplished through instrument sensing of reporter molecules resulting from Cas enzyme cleavage of target amplicons within the reaction chamber as the targets are amplified, thereby providing real-time detection.

At 414, the collected signal data is processed, and at 416, the diagnostic results provided to a user. Results processing/interpretation may be carried out via the instrument, or via an accompanying product application compatible with mobile devices or computers. The application may provide the user with a concise report regarding the results, and may even suggest a follow up screen or test if necessary. The application may also provide the user with product information, instructions for use, customer service, etc. The results are compatible with telemedicine practice, and the application can transmit test data to a patient's physician or a remote clinic for review and further consultation. In certain other embodiments, however, results may be indicated to the user via the test cartridge. For example, the test cartridge may comprise a visual indicator, e.g., a test window, wherein the results can be indicated via, e.g., color change, a change in a marked line or symbol, an appearance of a line or symbol (e.g., a plus or minus), etc. In certain embodiments, the test cartridge may comprise a small LCD display, which can provide an “answer” to the user, such as “yes” or “no.”

### EXAMPLE EMBODIMENTS

Embodiment 1: A point-of-care assay system for detection of high-risk human papillomavirus strains (hr-HPV) and HPV-related disease.

Embodiment 2: The assay system of Embodiment 1, further configured to screen for evidence of cervical intraepithelial neoplasia and cervical cancer.

Embodiment 3: The assay system of Embodiment 2, configured to employ CRISPR-Cas engineered products, isothermal amplification, and lateral flow detection to provide a robust, field deployable test that can be carried out by a professional or non-professional user within a variety of settings.

Embodiment 4: The assay system of one of Embodiments 1-3, further configured to detect monoplex or multiplex DNA and/or mRNA biomarkers providing evidence of infection with hr-HPV and HPV-related disease.

Embodiment 5: The assay system of one of Embodiments 1-4, configured to employ one or more CRISPR-Cas enzymes along with specific crRNA and/or trRNA (sgRNA) to detect both DNA and RNA biomarkers.

Embodiment 6: The assay system of Embodiment 5, configured to leverage multiple Cas enzymes for their inherent property to cleave non-specific or collateral ssDNA or ssRNA probes.

Embodiment 7: The assay system of Embodiment 1 or 5, wherein the final reaction/reporter products can be detected on lateral flow formats.

Embodiment 8: The assay system of Embodiment 1, configured to detect DNA evidence originating from up to 14 or more high-risk strains of HPV that may are associated with neoplasia or cancer of anogenital and head and neck cancers.

Embodiment 9: The assay system of one of Embodiments 1-8, configured to detect evidence of RNA expression of HPV oncogenes E6 and/or E7 from one or more high risks HPV strains.

Embodiment 10: The assay system of one of Embodiments 1-8, configured to detect RNA expression of biomarkers reflecting critical changes in patient cells associated with persistent, host genome integrated HPV.

Embodiment 11: The assay system of one of Embodiments 1-8, configured to detect biomarker(s) providing evidence of survival risk in identified HPV positive precancer or cancerous lesions.

Embodiment 12: The assay system of Embodiment 3, further configured to provide rapid amplification of low-copy target RNA or DNA biomarkers using low-temperature, robust and easily deployable methods.

Embodiment 13: The assay system of Embodiment 12, wherein the target amplification method is dependent upon recombinase polymerase and/or loop-mediated or reverse transcriptase loop-mediated amplification.

Embodiment 14: The assay system of one of Embodiments 1-13, further comprising a collection vial/device that accepts specimens such as liquid cervical samples, saliva, dry or wet swabs, biopsy tissue, blood, urine or other.

Embodiment 15: The assay system of one of Embodiments 1-14, further configured to accept and process cervical cellular material for evidence of HPV infection and disease.

Embodiment 16: The assay system of one of Embodiments 1-3 and 15, further configured to provide for sample preparation and processing within the sample collection vial and or an accompany test vial/cartridge.

Embodiment 17: The assay system of claim 16, further comprising a test vial/cartridge configured to accept specific physical and chemical inputs, to facilitate process activation and completion.

Embodiment 18: The assay system of Embodiment 17, further comprising membrane and housing units that fit into one or more openings in the cap of the test/vial cartridge in one orientation and provide for distinct processes to be completed.

Embodiment 19: The assay system of Embodiment 18, wherein the membrane is bifurcated and surface modified at the collection zone to enrich for DNA or RNA targets and to ensure binding of desired molecules within the bifurcated region of the membrane while inhibiting movement or binding to other regions of the membrane.

Embodiment 20: The assay system of Embodiment 19 wherein the membrane is comprised of one or more materials favorable for nucleic acid binding, such as but not limited to cellulose, silica, diatoms, PES, etc.

Embodiment 21: The assay system of Embodiment 18, therein the housing is constructed of rigid polymeric material that contains inner and outer sections, the inner section containing a membrane and being designed to slide within the outer section of the housing by virtue of depressing a spring-loaded handle on the distal end relative to the collection/binding zone. Depressing the spring handle distends the membrane binding/collection zone, below the body of the housing allowing it to contact with the bottom of a reaction chamber and/or solutions within the chamber located directly below the membrane housing.

Embodiment 22: The assay system of one of Embodiments 18 and 21, further comprising a housing in which the proximate end of the outer housing, relative to the membrane collection zone is fitted with an electrically conductive material that runs through the housing, such that the conductive material contacts electrical contacts in the test vial/cartridge completing an electrical circuit.

Embodiment 23: The assay system of Embodiment 18, wherein the vial/cartridge comprises inner rails, one or more reaction chambers, and cap, the cap containing one more openings into which a housing in claim 18 containing a specific membrane can be inserted, and the vial/cartridge designed with inner rails to accept and guide the housing ensuring the housing and membrane are positioned such that the housing rests just above specific internal chamber(s) of the test vial when the spring handle is depressed the membrane is distended entering the specific chamber.

Embodiment 24: The assay system of Embodiment 23, wherein the test vial/cartridge comprises reaction chambers containing rinse solutions and or lyophilized reagents for specific reactions and which may be sealed to prevent fluid loss or evaporation prior to use.

Embodiment 25: The assay system of Embodiment 21 or 22, wherein the membrane/housing contains a rigid point on the exterior section of the unit such that when fully inserted in the vial/test cartridge the rigid point ruptures a sealant film covering a buffer storage chamber, initiating flow of buffer(s) stored within the vial into a reaction chamber to solubilize lyophilized material necessary to support various reaction and or processes.

Embodiment 26: The assay system of one of Embodiment 21-23, wherein the vial/test cartridge comprises a base that is fitted with a disposable battery that is connected to an electrical circuit within the unit providing power to specific reaction chamber(s) in the vial used for target amplification or other processes requiring elevated solution temperatures.

Embodiment 27: The assay system of one of Embodiments 18, 21-23, and 25, wherein the housing comprises a lateral flow membrane such that the tip of the housing/membrane rests directly within the vial/cartridge chamber when the housing is fully inserted into the cartridge that has been configured and/or surface modified to support the capillary flow of a solution and materials therein, and which may contain specific molecules predeposited at distinct points along the solution path in order to concentrate reporter molecules generated in reaction chamber(s).

Embodiment 28: The assay system of Embodiment 27, wherein the lateral flow membrane comprises multiple lanes to distinguish conditions/results presented from one lane to the next, each providing distinct information.

Embodiment 29: The assay system of Embodiment 27 or 28, wherein a housing for the lateral flow membrane covers some regions of the membrane but provides openings for manually viewing results or recording an image of the results with a portable device such as a smartphone, tablet, etc.

Embodiment 30: The assay system of one of Embodiments 27-29, wherein the housing comprises external markings such as a bar code or other identifier to record test type, unit manufacture information, etc., when an image is captured.

Embodiment 31: The assay system of Embodiment 1, further comprising a sample vial configured to accept cellular specimens such as cervical specimens that have been collected by patient or clinician and which are deposited into the vial, which further contains a buffer for initial processing of specimen in preparation for transfer to the vial/test cartridge.

Embodiment 32: The assay system of Embodiment 31, wherein the buffer in the sample vial is configured to lyse whole cells that may be present in the sample, while preserving the integrity of nucleic acids contained therein. Such solution may be comprised of but not limited to chaotropic solution, detergent, buffers, etc.

Embodiment 33: The assay system of Embodiment 31 or 32, wherein the sample vial comprises a sealable opening on the vial cap, which when unsealed provides a port for entry of a membrane/housing unit designed to absorb/bind nucleic acids in the solution and serve as a means for transfer to the test cartridge.

Embodiment 34: The assay system of Embodiment 1, further comprising a disposable device for self-collection of a cervical specimen that facilitates patient self-collection of a specimen adequate for cervical screening, without the need for a clinic, clinician, or speculum.

Embodiment 35: The assay system of Embodiment 1, further comprising a software application for portable devices that can be used to capture image/unit data, perform and report results to user, as well as serve as a user interface for IFU, transmittal of data to physician/clinic, and/or provide company and test information.

Embodiment 36: The assay system of Embodiment 1, further comprising a single use, disposable microfluidic cartridge comprising one or more chambers for sample input, processing, and results reporting, and a portable bench-top instrument into which the cartridge is inserted, providing power, processing support, and a user interface.

Embodiment 37: The assay system of Embodiment 36, wherein the cartridge is constructed of multilayered polymeric materials, and contains one or more chambers for sample input/lysis, target capture, and CRISPR-based detection. In some embodiments, the cartridge comprises one chamber for carrying out sample input/lysis, target capture, and CRISPR-based detection.

Embodiment 38: The assay system of Embodiment 37, wherein target detection is accomplished via instrument-based sensing of fluorescent signals generated from CRISPR-directed cleavage of probes, the probes labeled with a specific quencher and a fluorophore and contained within a specific cartridge reaction chamber, the cartridge designed as a single-use, disposable unit barcoded with test/user information for tracking within inventory, LIS, and electronic record systems.

Embodiment 39: The assay system of Embodiment 36, further configured to accept a cervical cellular specimen in a preservative or dry swab format, the sample introduced into the cartridge lysis chamber(s) containing lyophilized or solution-based lysis buffer and poly-dT magnetic beads to lyse cells and facilitate capture of target nucleic acids.

Embodiment 40: The assay system of Embodiment 36, wherein the cartridge is inserted into an accompanying instrument, thereby controlling sample/stage residence time, temperature, buffer inputs, sample removal, and reagent flow. Following lysis and wash of nucleic acid targets captured on surface modified magnetic beads that have been transferred to the cartridge reaction chamber containing required reagents for RT-LAMP amplification and CRISPR detection in lyophilized format, the instrument directs the movement of fluids into/out of the reaction chamber to solubilize reagents and initiate reactions. Upon CRISPR recognition of target amplicons and CRISPR Cas12b-induced cleavage of fluor-quencher labeled probe(s) for specific targets, a fluorescent signal is generated and collected through instrument sensors. Algorithms embedded within the instrument are employed to calculate signal levels detected relative to desired thresholds for positive/negative and control values. Results are displayed in a specific results section of the cartridge, thereby providing ease of interpretation, while also being maintained in greater detail within the data stored directly on the instrument.

Embodiment 41: The assay system of Embodiment 39, wherein the instrument may be powered via AC or DC power systems, and wherein the instrument further comprises internet connectivity via Bluetooth, wireless connections, or direct connections for updates, data transfer, and user interface.

Embodiment 42: The assay system of any one of Embodiments 36, 37 or 40, wherein signals indicative of target detection are generated from detection probes labeled with fluorophores, with or with quenchers such as but not limited to FAM/BHQ, or enzymes capable of generating electrochemical species in the presence of a specific chemistry, such as but not limited to horseradish peroxidase and tetramethylbenzene, wherein said signals are collected via instrument sensing.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What we claim:

1. A point-of-care (POC) assay system for detection of high-risk human papillomavirus strains (hr-HPV) and HPV-related disease, the system comprising:
a test cartridge comprising:
a plurality of oligonucleotide primers for amplification of a target HPV oncogene sequence or hr-HPV marker sequence;
an endonuclease; and
a guide RNA configured to bind to and direct the endonuclease to the target HPV oncogene sequence or hr-HPV marker sequence.

2. The assay system of claim 1, wherein the target HPV oncogene or hr-HPV marker is an indicator of cervical intraepithelial neoplasia and cervical cancer.

3. The assay system of claim 1, wherein the endonuclease comprises a Cas endonuclease.

4. The assay system of claim 3, wherein the Cas endonuclease comprises a Cas9 endonuclease, a Cas12 endonuclease, a Cas13 endonuclease, or variants thereof.

5. The assay system of claim 1, wherein the target HPV oncogene sequence or hr-HPV marker sequence comprises an L1 sequence, an L2 sequence, an E6 sequence, or an E7 sequence.

6. The assay system of claim 1, wherein the target HPV oncogene sequence or hr-HPV marker sequence comprises a p16 sequence, an ERK-1 sequence, a hTert sequence, a LR-67 sequence, a MMP-2 sequence, a Nf-Kβ sequence, a nm23-H1 sequence, a PCNA sequence, a survivin sequence, a Topo-2α sequence, a VEGF-C sequence, or a cytokeratin 17 (K17) sequence.

7. A point-of-care (POC) lateral flow assay system for detection of high-risk human papillomavirus strains (hr-HPV) and HPV-related disease, the system comprising:
a dipstick, comprising:
a substrate for capture of nucleic acids comprising a target HPV oncogene sequence or hr-HPV marker sequence; and
a sample vial comprising:
a reaction chamber or cartridge configured to receive the dipstick, the reaction chamber comprising:
an oligonucleotide primer for amplification of a target HPV oncogene sequence or hr-HPV marker sequence;
an endonuclease; and
a guide RNA configured to bind to and direct the endonuclease to the target HPV oncogene sequence or hr-HPV marker sequence.

8. The assay system of claim 7, wherein the target HPV oncogene or hr-HPV marker is an indicator of cervical intraepithelial neoplasia and cervical cancer.

9. The assay system of claim 7, wherein the endonuclease comprises a Cas endonuclease.

10. The assay system of claim 9, wherein the Cas endonuclease comprises a Cas9 endonuclease, a Cas12 endonuclease, a Cas13 endonuclease, or variants thereof.

11. The assay system of claim 7, wherein the target HPV oncogene sequence or hr-HPV marker sequence comprises an L1 sequence, an L2 sequence, an E6 sequence, or an E7 sequence.

12. The assay system of claim 7, wherein the target HPV oncogene sequence or hr-HPV marker sequence comprises a p16 sequence, an ERK-1 sequence, a hTert sequence, a LR-67 sequence, a MMP-2 sequence, a Nf-Kβ sequence, a nm23-H1 sequence, a PCNA sequence, a survivin sequence, a Topo-2α sequence, a VEGF-C sequence, or a cytokeratin 17 (K17) sequence.

13. A method of detecting high-risk human papillomavirus strains (hr-HPV) and HPV-related disease, comprising:
lysing a cervical cell sample in a liquid-based medium;
capturing a nucleic acid of the lysed cervical cell sample on a substrate;
amplifying the captured nucleic acid;
cleaving the amplified nucleic acid with an endonuclease, the cleaving producing a detectable signal; and
detecting the detectable signal to identify the cleaved nucleic acid.

14. The method of claim 13, wherein the nucleic acid comprises a target HPV oncogene sequence or a hr-HPV marker sequence.

15. The method of claim 14, wherein the target HPV oncogene sequence or hr-HPV marker sequence comprises an L1 sequence, an L2 sequence, an E6 sequence, or an E7 sequence.

16. The method of claim 14, wherein the target HPV oncogene sequence or hr-HPV marker sequence comprises a p16 sequence, an ERK-1 sequence, a hTert sequence, a LR-67 sequence, a MMP-2 sequence, a Nf-Kβ sequence, a nm23-H1 sequence, a PCNA sequence, a survivin sequence, a Topo-2α sequence, a VEGF-C sequence, or a cytokeratin 17 (K17) sequence.

17. The method of claim 13, wherein the endonuclease comprises a Cas endonuclease.

18. The method of claim 17, wherein the Cas endonuclease comprises a Cas9 endonuclease, a Cas12 endonuclease, a Cas13 endonuclease, or variants thereof.

19. The method of claim 13, wherein the substrate comprises a modified magnetic bead of a test cartridge.

20. The method of claim 13, wherein the nucleic acid is amplified via polymerase chain reaction (PCR), loop mediated isothermal amplification (LAMP/RT-LAMP), recombinase polymerase amplification (RPA), nucleic acid sequence-based amplification (NABA), helicase-dependent amplification (HDA), strand displacement amplification (SDA), exponential amplification (EXPAR), rolling circle amplification (RCA), or nicking extension amplification reaction (NEAR).
